(19) United States
(12) Patent Application Publication
(10) Pub. No.: US 2005/0197865 A1
(43) Pub. Date: Sep. 8, 2005

(54) Physiologic inference monitor

(76) Inventor: Desmond Jordan, Hoboken, NJ (US)

Correspondence Address: LESLIE GLADSTONE RESTAINO;BROWN RAYSMAN MILLSTEIN & STEINER LLP, 163 MADISON AVENUE, PO BOX 1989, MORRISTOWN, NJ, 07962-1989, US

(21) Appl. No.: 10/795,724

(22) Filed: Mar. 5, 2004

(51) Int. Cl. G06F 17/60; A61B 19/00

(52) U.S. Cl. 705/3; 128/898; 434/232

(57) ABSTRACT

The present invention provides methods, systems, and computer programs for inferring a patient's clinical status in the course of treatment. The method includes the steps of accessing a patient's data, such as data collected prior, during, and/or after the treatment, and identifying from the patient's data abnormal events occurring in the course of the treatment. The abnormal events are generally identified by applying a scoring system for inferring a patient's clinical status. This scoring system includes a plurality of scoring schemes applicable at different times with respect to at least one milestone event.

### BACKGROUND OF THE INVENTION

The present invention relates to patient monitoring systems. In particular, the present invention relates to systems for monitoring a patient's operative course and clinical status, and for briefing subsequent caregivers regarding the patient's treatment and clinical status.

In advance of a patient's arrival at a Cardio Thoracic Intensive Care Unit (“CTICU”), information regarding the patient's operative course and clinical status must be made available to the CTICU medical team, so that the team may provide prompt and appropriate therapy should problems arise. Some information regarding the patient may be provided during the operation by telephone; however, this information is typically cursory in nature. The majority of the patient information is typically conveyed in a post-operative summary or briefing, which is usually given orally to a CTICU physician by an operating room (“OR”) physician.

Due to the time constraints under which caregivers work, the structure, organization, and amount of information, and the varied importance of the information to the briefing physician, patient-related information is not consistent for each patient. Moreover, the information conveyed to the CTICU medical team is not readily accessible to subsequent caregivers that were not present at the post-operative briefing; thus, subsequent caregivers must independently review a patient's records, and independently assess the patient's condition. This independent review of the patient's status, both during and after the operation, leads to a lack of medical care continuity, loss of information transfer, and increased medical judgment errors.

Accordingly, there exists a need for methods for briefing subsequent caregivers regarding a patient's operative course, and regarding the clinical operative and/or post-operative status, that are more efficient than the present methods. There is also a need for methods for briefing subsequent caregivers with information that is consistent for each patient or all patients.

### SUMMARY OF THE INVENTION

The present invention generally provides methods, systems, and computer programs for making inferences from patient data collected in the course of a treatment, and for briefing subsequent caregivers regarding a patient's therapeutic treatment, severity of condition, and clinical status, which overcome the deficiencies in the art relating to inferring a patient's clinical status and briefing subsequent caregivers thereof. Although the present invention may be described, by way of example, in relation to particular surgical operations, such as cardiac surgeries, and particular clinical staff, such as the CTICU medical staff, it is understood that the present invention may be used in a variety of therapeutic and non-therapeutic treatments to brief a variety of medical and non-medical staff, and, therefore, is not limited thereto.

In one aspect of the invention, methods and corresponding systems are provided for inferring a patient's clinical status, in the course of a treatment, by accessing a patient's data, and identifying from the patient's data at least one abnormal event occurring within the course of the treatment. Abnormal events are generally identified by applying a scoring system for inferring a patient's clinical status, wherein the scoring system includes a plurality of scoring schemes, including a first scoring scheme for identifying from patient data abnormal events occurring prior to at least one milestone event, and a second scoring scheme for identifying from patient data abnormal events occurring after the at least one milestone event.

Various types of patient data may be used as a basis for the inference or identification of the abnormal events occurring during the course of the patient's treatment. In one embodiment, in which the patient's data include pre-operative data, such as demographic data and clinical data, the identification of the at least one abnormal event is based, at least in part, on the patient's pre-operative data. In another embodiment, the patient's data include operative data, such as data concerning a patient's vital signs, anesthetics delivered, ventilation parameters, drugs delivered, laboratory results, intravenous lines attached to the patient, devices used, and beginning and start times of events. Various types of abnormal events that occur during the course of treatment may be identified on the basis of this operative data, including abnormal events related to hemodynamics, abnormal events related to laboratory results, and abnormal events indicative of the severity of a patient's condition.

In another embodiment, at least one of the scoring schemes assigns a score to abnormal patient data that is based on the severity of the patient's condition, as reflected in the patient's data. With regard to abnormal hemodynamic events, which are typically based on a plurality of consecutively-repeated patient readings, the at least one abnormal event may be identified by applying temporal abstraction to the patient's operative data that have been scored in accordance with the scoring system for inferring a patient's clinical status. Temporal abstraction may include the step of applying a sliding scale average over a window of a predefined number of consecutively-occurring operative data that have been scored in accordance with the scoring system for inferring a patient's clinical status.

In another embodiment, the patient's data is collected in connection with a surgery that includes a cardiac bypass. In this instance, the at least one milestone event is the cardiac bypass, the first scoring scheme is applied to data occurring prior to the cardiac bypass, and the second scoring scheme is applied to data occurring after the cardiac bypass.

In another embodiment, identification of at least one abnormal event is based, at least in part, on at least one of: a duration of the treatment or a portion thereof, whether blood products were administered, quantities of blood products administered, whether drugs were administered, quantities of drugs administered, or a combination thereof. In another embodiment, the method of the present invention further comprises identifying drugs and quantities thereof administered in connection with an abnormal event, and noting this information in a briefing that is prepared in accordance with the present invention. Similarly, in yet another embodiment of the present invention, the method further comprises linking a milestone event with one or more abnormal events occurring within a predefined period of time from the milestone event, and nothing, in a briefing, whether the abnormal events occurred within the predefined period of time of the milestone event.

In another embodiment, a patient's treatment is monitored, and an operation status monitor interface is displayed or caused to be displayed. The interface may include a patient summary for at least one patient undergoing a treatment, and may also include a graphical presentation of the patient's treatment status in a time-dependent format. Upon completion of the patient's treatment, a briefing may be produced; the briefing may include any abnormal events identified in connection with the patient's treatment, and may be generated for a target audience. The incidence of the patient from the operation status monitor interface then may be transferred to a patient post-operative briefing interface, which indicates that a briefing for the patient is available for review. In one embodiment, the patient post-operative briefing interface includes a graphical presentation, in a time-dependent format, of at least one of an abnormal event identified in connection with the patient's treatment and a milestone event.

In another aspect of the invention, methods and corresponding systems are provided for inferring a patient's clinical status in the course of a treatment, by accessing a patient's pre-operative data and operative data; identifying from the patient's pre-operative and operative data at least one abnormal event indicative of the severity of a patient's condition, by applying a scoring system for inferring a patient's clinical status; and producing a briefing that includes at least one abnormal event identified in connection with the patient's treatment. The scoring system includes a plurality of scoring schemes, including a first scoring scheme for identifying from patient data at least one abnormal event occurring prior to at least one milestone event, and a second scoring scheme for identifying from patient data at least one abnormal event occurring after the at least one milestone event.

In another aspect of the invention, methods and corresponding systems are provided for inferring a patient's clinical status in the course of a treatment, by monitoring a patient's treatment; displaying an operation status monitor interface, which includes a patient summary for at least one patient undergoing a treatment; accessing a patient's data, including pre-operative data and operative data; identifying abnormal events from the patient's pre-operative and operative data, by applying a scoring system for inferring a patient's clinical status; automatically producing, at the end of the treatment, a briefing that includes at least one abnormal event identified in connection with the patient's treatment; and transferring an incidence of the patient from the operation status monitor interface to a patient post-operative briefing interface which indicates that a briefing for the patient is available for review. In one embodiment, the briefing is a multimedia briefing, wherein the multimedia briefing is made available on a patient post-operative briefing interface that includes controls for a user to control presentation of the multimedia briefing.

Additional aspects of the present invention will be apparent in view of the description which follows.

### BRIEF DESCRIPTION OF THE FIGURES

FIG. 1 is a flow diagram of a method for briefing subsequent caregivers regarding a patient's operative course and clinical status, according to one embodiment of the present invention.

FIG. 2 is a block diagram of a system for briefing subsequent caregivers regarding a patient's operative course and clinical status, according to one embodiment of the invention.

FIG. 3 is an operation status monitor interface screen, according to one embodiment of the invention.

FIG. 4 is a post-operative briefing interface screen, according to one embodiment of the invention.

FIG. 5 is a post-operative briefing interface screen, according to another embodiment of the invention.

### DETAILED DESCRIPTION OF THE INVENTION

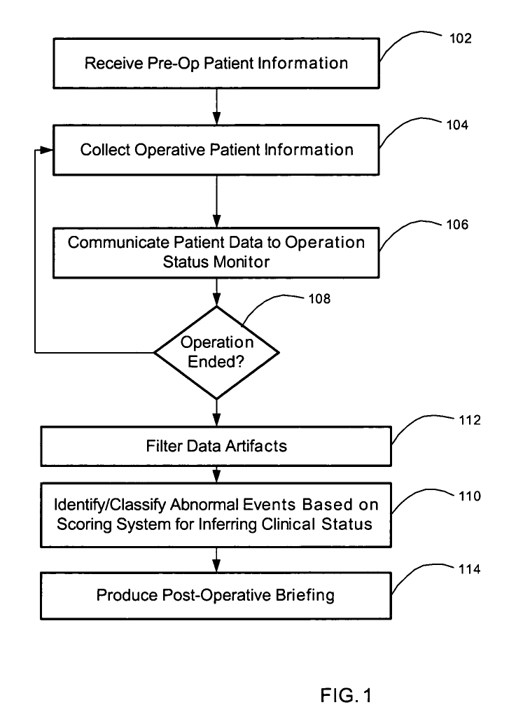

Referring to FIG. 1, a method for briefing subsequent caregivers regarding a patient's operative course and clinical status, in accordance with one embodiment of the present invention, begins at step 102 by receiving a patient's pre-operative information. The term “operative” is used herein generally to denote a therapeutic treatment, whether surgical, medical, or otherwise. The type and quantity of pre-operative information will vary depending on the type of medical treatment for which a briefing, in accordance with the present invention, will be prepared. The pre-operative information will generally include identification and/or demographic data regarding the patient, such as the patient's name, address, gender, age, weight, identification number(s), etc. The pre-operative information may also include pre-operative clinical data, such as the patient's vital signs, relevant allergic reactions, medications, prosthetics, pre-existing medical conditions, relevant diagnoses, the type of procedure recommended, an indication as to whether the patient arrived through emergency, or whether the recommended procedure is being redone, etc. The pre-operative data may then be stored in a patient records database that is accessible for later use. The pre-operative information may be obtained at any time prior to the medical treatment (e.g., in connection with an office visit with the patient's primary physician, in connection with a hospital visit, or a combination thereof).

During the course of a treatment, such as a surgery, at least some patient operative information is collected at step 104, using a data acquisition system such as the LifeLog data acquisition system. The data acquisition system automatically and repeatedly captures and records operative data from various medical devices, such as Hewlett Packard Merlin monitors, Ohmeda anesthesia machines, and saturation monitors. The data acquisition system preferably collects a patient's operative data, such as the patient's vital signs, inhaled anesthetics, and ventilation parameters. In one embodiment, the data acquisition system preferably includes an interface for a user manually to enter operative data, including pre- and post-operative data, such as data regarding bolus drugs delivered, pre- and post-operative drugs delivered, laboratory results, intravenous lines attached to the patient, information about devices such as pacemakers, data from echocardiograms, etc. Surgical events or milestones, such as the time of intubation, skin incision, and start and stop of a bypass, for instance, or any other predetermined or user-defined event, may also be entered manually. The operative information may be stored in the patient records database for use by the inference monitor or engine in assessing the patient's clinical status, as described herein.

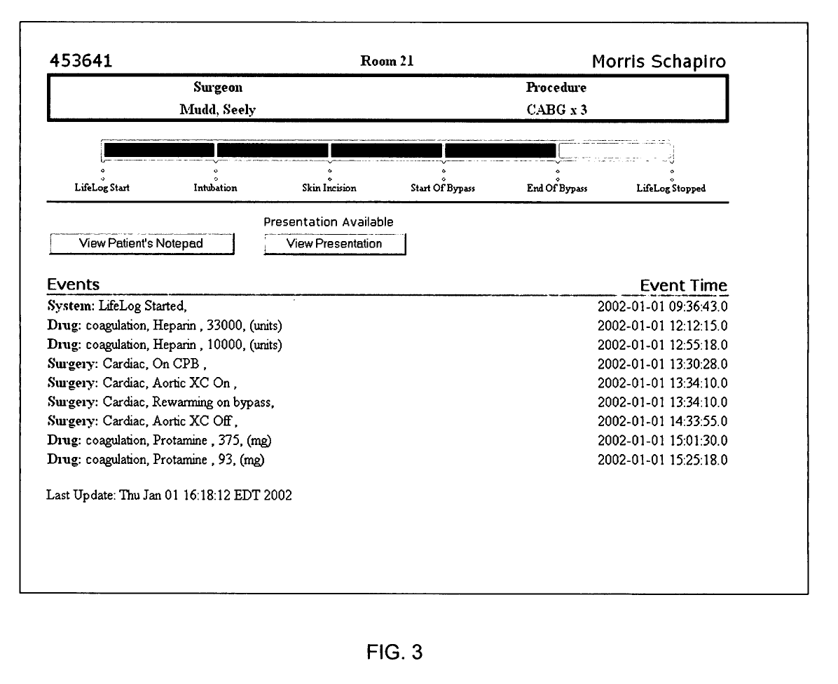

In one embodiment of the present invention, some or all of the patient pre-operative and/or operative data is communicated at step 106 to an operation status monitor, which monitors one or more ongoing operations in the operating room (OR). The operation status monitor generally provides a corresponding interface (e.g., a graphic user interface), such as the interface shown in FIG. 3, which includes patient summaries for patients undergoing treatment. The patient summaries may include such information as the name of the patient, the type of surgery or treatment, the then-current operation status (e.g., in a timeline format), lists of events that occurred during the course of the treatment, etc. In one embodiment, the operation status monitor is adopted to identify an abnormal patient status or event in connection with a physiologic inference monitor or engine, as described herein. This abnormal status may be displayed in the operation status monitor interface screen.

If it is determined, at step 108, that the operation has ended, then in one embodiment of the present invention the system will, at step 114, produce a post-operative briefing, preferably automatically, for subsequent caregivers. In instances where a caregiver must act quickly on the basis of a patient's clinical status, a succinct overview, highlighting important events regarding the patient's clinical status, is more efficient than an exhaustive log of the patient's vital signs, procedures, and laboratory results. For example, a single sentence that indicates that an episode of hypertension occurred during bypass surgery could summarize effectively what would otherwise be an overwhelming number of low-level raw blood pressure readings gathered during the surgery (which could include over 1,000 readings for an average five-hour bypass surgery). Accordingly, the patient briefing summarizes detailed patient data, and identifies abnormal patient status(es) based on patterns of data in the patient's records (e.g., in the pre-operative, operative, and post-operative information/data), as recognized by the inference monitor or engine. In one embodiment, the patient briefing is a multimedia briefing, which includes graphic and audio representations of the summarized data.

In another embodiment of the present invention, the inference monitor identifies and/or classifies abnormal patient events by scanning a patient's records for relevant data, such as pre-operative and operative patient information, and applying one or more inference rules at step 110. For example, with respect to a cardiac bypass surgery, the inference engine may apply the inference rule or rules to the patient's vital sign data, such as heart rate and blood pressure readings, and laboratory results. The vital signs are generally sampled by the data acquisition system at about 50-second intervals, and about 1-10 laboratory tests are made before and after the bypass surgery.

In a further embodiment, the inference monitor identifies and/or classifies abnormal events that occurred at or about certain milestones in the therapeutic treatment. A “milestone” or “milestone event” refers herein to a distinguishable event in the course of a patient's treatment. It is understood that various types of milestones may be either predefined or user-defined, based on the type of treatment being offered. For instance, with regard to bypass surgery, the milestones may be critical surgical points, such as the points of induction or intubation, skin incision, start of bypass, end of bypass, etc. In this instance, it may be desirable for the inference engine to apply the inference rule or rules to operative patient data that have been collected within a predefined window, such as a 20-minute window, before and/or after any one or more of the milestones.

The inference engine may also identify abnormal events in light of pre-operative information, such as demographic data (e.g., the patient's age, gender, weight, etc.) and pre-operative clinical data (e.g., the patient's vital signs, relevant allergic reactions, medications, prosthetics, preexisting medical conditions, relevant diagnoses, type of procedure suggested), and in light of operative data, such as the time interval of the events (e.g., between start and stop times) and the drugs that were administered during the operation. Typical drugs include pressors, such as phenylephrine, ephedrine, etc., and depressors, such as esmolol, nitroglycerine, etc.

In one embodiment of the present invention, the inference engine is adopted to identify a plurality of classes of abnormal events, including, without limitation, those relating to hemodynamics, those indicated by laboratory results, and those indicative of the severity of a patient's condition. By way of example, hemodynamic inferences identify episodes of hypotension, hypertension, bradycardia, and tachycardia. Laboratory inferences identify acidosis, alkalosis, hypercardia, hypoxia, low saturation, hyponatremia, hypernatremia, hypokalemia, hyperkalemia, hypocalcemia, hypercalcemia, anemia, hypoglycemia, and hyperglycemia, for example. Inferences identifying events indicative of the severity of a patient's condition, include, without limitation, duration of treatment, the type of procedure, demographics, blood products given, and bolus drugs or drips.

Various techniques may be used to identify abnormal events from a patient's data. In one embodiment of the present invention, the system applies one or more inference rules that identify abnormal events based on a scoring system for inferring a patient's clinical status based on abnormal event thresholds for the scores. In another embodiment, the scoring system includes a plurality of independent scoring schemes, which include at least one scoring scheme applicable prior to a milestone, and at least one scoring scheme applicable after the milestone. A single threshold, common to the plurality of scoring schemes, may be applied to infer the patient's clinical status; a plurality of thresholds, corresponding to each of the plurality of schemes, may also be applied. By way of example, a scoring system may comprise a first scoring scheme for identifying from patient data at least one abnormal event occurring prior to at least one milestone event, and a second scoring scheme for identifying from patient data at least one abnormal event occurring after the at least one milestone event.

A scoring system in accordance with an exemplary embodiment of the present invention is provided in Appendix A, Tables A-J. It is understood that the inference monitor may apply various scoring systems without departing from the spirit of the invention, insofar as the scoring systems are designed to identify abnormal events from repeated measures of data, particularly operative data of the patient. Therefore, the present invention is not limited to any one particular scoring system.

In a further embodiment, the inference monitor scores abnormal readings based on a scoring system, wherein the scoring system assigns increasingly higher scores for abnormal readings showing increasingly greater severity of a patient's condition. The score may increase linearly or nonlinearly (such as exponentially, logarithmically, etc.) with increasing severity. For example, with respect to tachycardia, a score of 3 may be assigned to a heart rate reading that is less than 120 bpm, with a score of 10 for a heart rate reading that is equal to 120-130 bpm, and a score of 20 for a heart rate reading that is above 130 bpm. From this illustrative example, it can be seen that increasingly higher scores are assigned to abnormal readings showing increasing severity of the tachycardia. In another embodiment, for bypass surgeries, a dual scoring scheme is applied, wherein one scoring system is applied to at least part of the data occurring prior to the bypass or pre-bypass, and another scoring system is applied to data occurring after the bypass or post-bypass.

In another embodiment of the present invention, the inference engine identifies abnormal events (i.e., the severity of the patient's condition) based, at least in part, on the duration of the treatment or a portion thereof. The inference engine may make the inference based on a scoring system that assigns an increasingly higher score for an increasingly higher (increasingly greater severity) duration. For example, and with reference to Table A and Table B, a score for a bypass surgery may be assigned for the duration of the treatment (from induction to end of bypass) and/or for the duration of the bypass (from start to end of bypass). As can be seen in Table A, scores of 1, 2, 5, 8, and 10 are assigned to bypasses of 60, 90, 100, 150, and 180 minutes, respectively. A threshold score of 5 may be assigned as abnormal, and, at least with respect to the post-operative briefing, durations exceeding the threshold may be flagged as abnormal events, indicative of the severity of the patient's status, that should be included in the briefing. The score may also be factored into, or weighed in connection with, an inference of abnormal hemodynamic or laboratory events. The inference engine may similarly infer the severity of the patient's condition with regard to the type of procedure; the patient's age, weight, and gender; an indication as to whether the patient arrived through the emergency room, whether the procedure is a repeat procedure, whether anesthesia was used, etc.

In one embodiment, the inference engine identifies an abnormal event (i.e., the severity of the patient's condition) based, at least in part, on an indication as to whether or not blood products were administered and/or the quantity of the blood products administered. The inference engine may make the inference based on a scoring system that assigns a score that is increasingly higher with increasing quantity of blood products. With regard to bypass surgery, and with reference to Table C, an indication that particular types of blood products were given, and blood products exceeding a threshold quantity, are flagged as abnormal events that are indicative of the severity of the patient's condition, and should be included in the post-operative briefing. The inference engine may similarly infer the severity of the patient's condition based on whether or not drugs were administered, and/or the quantity of the drugs administered, as shown in Tables D-F. The score may also be factored into, or weighed in connection with, an inference of abnormal hemodynamic or laboratory events.

Since hemodynamic inferences are based on a set of frequent readings over temporal intervals, temporal abstraction may be used to determine abnormality in this respect from the set of readings. For example, the inference rules concerning hypotension may classify an event as abnormal when blood pressure falls below 100 for 250 seconds (five 50-second intervals) or, with respect to a scoring system, when scores for hypotension exceed a threshold for five consecutive readings.

In another embodiment of the present invention, a sliding scale average is applied to a window of a predefined number of consecutive values (e.g., 5 consecutive readings of blood pressure and heart rate scores), thereby smoothing out temporal variations in data. If the average does not exceed the threshold, the inference engine drops the oldest value, and slides forward in time to add a new value. If the average meets the threshold, the start of an abnormal episode is recorded; the inference engine then continues calculating sliding averages across the window until the average returns to a normal value, marking the end of the episode. Once the time period for each episode has been calculated, the inference engine may record the drugs, and the amounts that were administered in connection with the abnormal episode, so that the briefing can describe treatment. In one embodiment, after all abnormal episodes have been found, the inference engine links each abnormal episode with each of the milestone events (e.g., the four critical time points—induction/intubation, skin incision, start of bypass, and end of bypass), noting whether the abnormal episode occurred within a window of about 20 minutes before or after each milestone.

In a further embodiment, the inference engine filters the data, at step 112, to remove artifacts therefrom, preferably prior to scoring. The term “artifacts” is used herein generally to denote false abnormal readings. For example, a spike may occur in heart rate or blood pressure, because of electric cautery, blood draws, catheter flushing, etc. To avoid making false inferences from artifacts, the system filters the data, preferably automatically, prior to inferring or otherwise identifying abnormal events, so as to retain only data in cases where values remain within valid ranges and where changes in one value (e.g., heart rate) are accompanied by an appropriate change in the other (e.g., blood pressure). For example, if a patient experiences a change in heart rate greater than 50, within a 50-second interval, the system retains the spike if there is a corresponding change of 10 in blood pressure. If blood pressure does not change, then the spike is replaced with the last good heart rate value. The reverse is also true: spikes in blood pressure are retained when accompanied by changes in heart rate. In another embodiment, the system filters data when: all three blood pressures (mean, systolic, and diastolic) are equal, any systolic blood pressure is greater than 250 psi, and both blood pressure and heart rate are zero. In the last instance, the zeros may be replaced by average heart rate and blood pressure, provided the patient is not currently on bypass.

In one embodiment of the present invention, the system separately inspects laboratory test data obtained before, during, and after bypass. The laboratory tests that are performed during bypass are not normally indicative of patient post-operative status; however, the data may be used by the inference engine to classify abnormal events in combination with hemodynamic inferences. Pre- and post-bypass laboratory result thresholds may be applied to determine whether or not the results are abnormal. The inferred information may then be stored in a patient records database, along with other data (e.g., demographics, medical history, and drugs given), to be used as the content for the post-operative briefing.

The post-operative briefing may then be produced at step 114. In one embodiment, the post-operative briefing is generated for a specific target audience. For instance, the post-operative briefing may be generated for a physician, resident, nurse, layperson, etc. The briefing may also be generated for particular departments, such as administration, cardiology, etc. Accordingly, some or all of the data or inferences may be omitted from the briefing, based on the identity of the targeted audience. For example, heart rate data/inferences prior to bypass may be omitted from a briefing prepared for nurses, whereas post-bypass heart rate data/inferences may be included in briefings targeted to cardiologists and nurses.

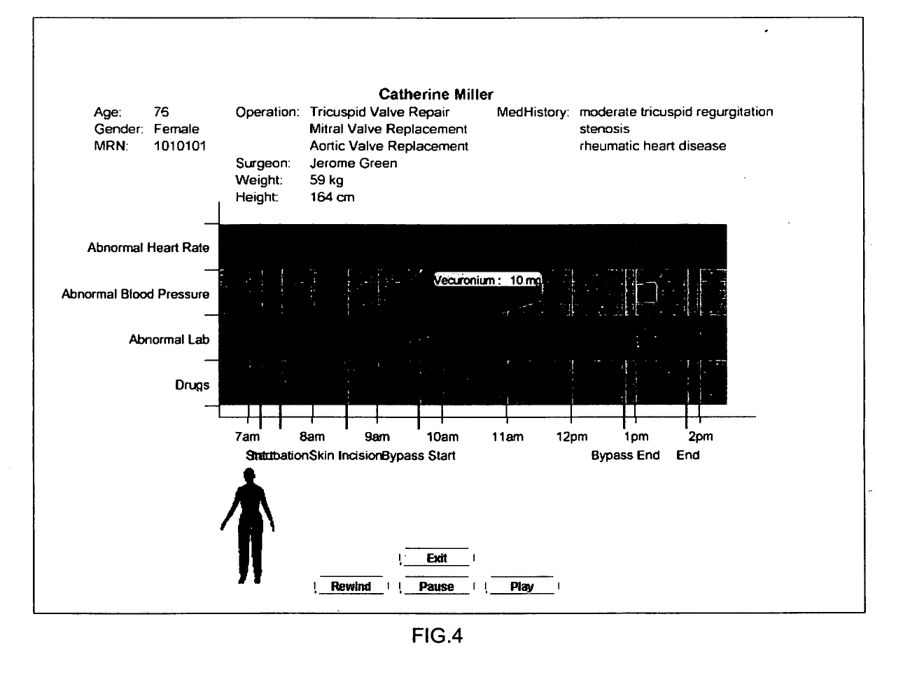

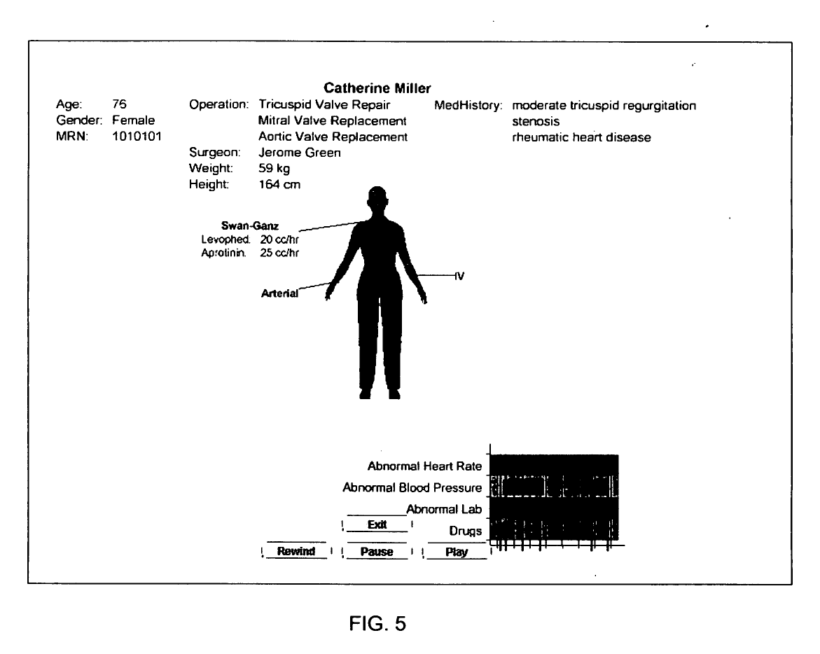

In yet another embodiment, the briefing is made available on an interface separate from the operation status monitor interface. At the end of an operation, the incidence of the patient on the operation status monitor interface is automatically transferred to a patient post-operative briefing interface (e.g., a graphic user interface), which indicates that a briefing for the patient is available for review. The patient post-operative briefing interface preferably includes controls therein, such as links or buttons, which allow a user to control the graphic presentation (e.g., to play, pause, stop, rewind, and advance the recitation), as shown in FIG. 4 and FIG. 5. In one embodiment, where a graphic presentation is provided, the interface screen includes therein a viewing section or window for displaying the presentation. The graphic presentation generally consists of graphics and audio that provide summarized information, including inferences, regarding the data that have been captured from the operating room during surgery. Typical presentations may last from 1-2 minutes per patient, depending on the quantity of information presented.

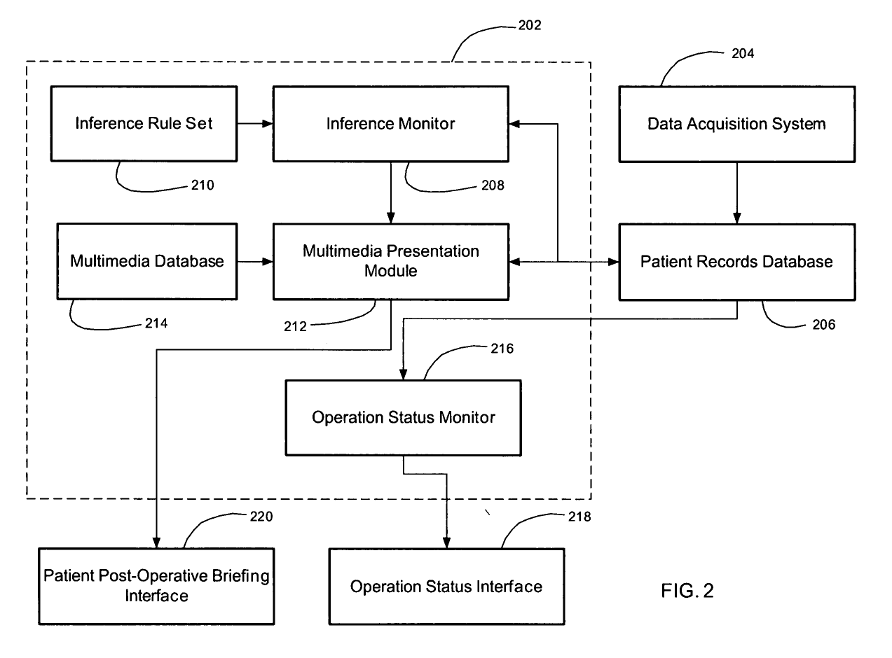

Referring to FIG. 2, a system for briefing subsequent caregivers on a patient's operative course and clinical status, according to one embodiment of the invention, includes at least one computing device 202, which includes an inference monitor 208. The inference monitor 208 is generally a software component that, when executed, is adopted to identify or classify abnormal events from patient data, by applying an inference rule or rules, as described above. Accordingly, the inference monitor 208 interfaces with a patient records database 206, which generally includes patient data, such as pre-operative, operative, and post-operative data, and an inference rule set 210. The inference monitor 208 stores the abnormal events inferences or classifications on the patient records database 206 for later use.

In one embodiment, the system includes a multimedia presentation module 212, which generally prepares a multimedia post-operative briefing that includes the inferences of abnormal events produced by the inference monitor 208. The multimedia presentation module 212 accesses a multimedia database 214, which includes graphic and audio data for the preparation of the post-operative briefing. Once the briefing is complete, the briefing or a plurality of briefings is/are stored on the patient records database 206 for presentation to subsequent caregivers. In a further embodiment, the multimedia presentation module 212 provides a post-operative briefing interface 220, which, as described above, includes therein links or buttons to facilitate control of the briefing presentation, as shown in FIG. 4. The interface 220 may be provided to a local user (e.g., with a display connected directly to the computing device 202), or to a second, remote computing device (not shown) that is connected to the computing device 202 over a communication network, such as a LAN, WAN, the Internet, etc.

Referring to FIG. 3, an operation status interface according to one embodiment of the invention includes a patient summary for at least one patient undergoing treatment. The patient summary generally includes the patient's name and information regarding the status of the treatment. The status of the treatment, for instance, may be presented in a timeline format, as shown, or in any other manner that permits presentation of information in a time-dependent format. The operation status interface may also include information regarding the physician performing the treatment, the type of procedure being performed, and a list of the events, preferably generated in real time. In one embodiment, the interface includes therein a link for displaying a post-operative interface screen, as described herein, when a post-operative briefing becomes available.

Referring to FIG. 4, a post-operative interface screen, according to one embodiment of the invention, includes therein a graphical representation of abnormal and/or other events on a timeline format. The graphical representation may also highlight the relevant milestone events, such as the start of intubation, skin incision, bypass start, bypass end, and end of procedure, as shown. The interface screen preferably includes therein buttons for controlling the presentation of the post-operative briefing, including, without limitation, buttons for playing, rewind, forwarding, pausing, and exiting from the presentation. The graphical representation may also include a status bar that highlights the timing of particular events on the timeline, and which may be dynamically controlled by the user to highlight other events and the details thereof. For example, the status bar indicates that anesthetics were administered between 7 a.m. and 8 a.m., and shows the particular types of anesthetics that were administered. Moving the status bar to the abnormal heart rate graphic between 12 p.m. and 1 p.m. would similarly result in presentation of the details of the abnormal heart rate.

In yet another embodiment, the computing device 202 includes an operation status monitor software component 216, as shown in FIG. 3. This software component monitors ongoing operations, and provides an operation status monitor interface 218, as shown in FIG. 3 and described above. The operation status interface 218 may be provided to local users or remote users, similar to the post-operative briefing interface 220.

While the foregoing invention has been described in some detail for purposes of clarity and understanding, it will be appreciated by one skilled in the art, from a reading of the disclosure, that various changes in form and detail can be made without departing from the true scope of the invention in the appended claims.

### APPENDIX A

TABLE A

| | | | Severity Scoring | Severity Scoring |
|---|---|---|---|---|
| Times | ID | Accessibility | Pre-Bypass | Post-Bypass |
| Start Time | ST | Administration | | |
| Induction Time | IT | Administration | | |
| Pre-bypass Time | PRT | Administration | | |
| (Start of Bypass) | | | | |
| Pos-t bypass Time | PBT | Administration | | |
| (End of Bypass) | | | | |
| Combinations | ST-IT | Administration | | |
| (Minutes) | | | | |
| | ST-PRT | Administration | | |
| | ST-PBT | Administration | | |
| | IT-PRT | Administration | | |
| | IT-PBT | Administration | 120 = 5 | |
| | | | 150 = 8 | |
| | | | 180 = 10 | |
| | PRT-PBT | Administration, | 60 = 1 | |
| | | Cardiology, | 90 = 2 | |
| | | Resident, Nursing | 120 = 5 | |
| | | | 150 = 8 | |
| | | | 180 = 10 | |

TABLE B

| | | | Severity Scoring | Severity Scoring |
|---|---|---|---|---|
| Demographics | ID | Accessibility | Pre-Bypass | Post-Bypass |
| Name | LAST NAME | Administration, | | |
| | FIRST NAME | Cardiology, | | |
| | | Resident, Nursing | | |
| Chart No. | MRN | Administration, | | |
| | | Cardiology, | | |
| | | Resident, Nursing | | |
| Procedure | CABG | Administration, | CABG1-3 = 1 | |
| | VALVE | Cardiology, | CABG4 = 5 | |
| | CABG-VALVE | Resident, Nursing | CABG5 = 10 | |
| | | | VALVE1 = 3 | |
| | | | VALVE2 = 10 | |
| | | | VALVE&CARB = 15 | |
| Age | AGE | Administration, | 65 = 2 | |
| | | Cardiology, | 70 = 3 | |
| | | Resident, Nursing | 75 = 5 | |
| | | | 80 = 10 | |
| | | | 85 = 15 | |
| Weight | WT | Administration, | 85 = 3 | |
| | | Cardiology, | 80 = 5 | |
| | | Resident, Nursing | 95 = 10 | |
| Sex | SEX | Administration, | FEMALE = 8 | |
| | | Cardiology, | | |
| | | Resident, Nursing | | |
| Emergency | ER | Resident | YES = 10 | |
| Redo | REDO | Administration, | YES = 15 | |
| | | Resident, Nursing | | |
| Anesthesia | ASA | | #4 = 10 | |
| | | | #5 = 20 | |
| | | | #6 = 30 | |

TABLE C

| | | | | Severity | Severity |
|---|---|---|---|---|---|
| Blood | | Pre-bypass | Post-bypass | Scoring | Scoring |
| Products | ID | Accessibility | Accessibility | Pre-Bypass | Post-Bypass |
| Cell Saver | CSB | | Cardiology, | Yes = 14 | #3 = 10 |
| Blood | | | Resident, | | #5 = 20 |
| | | | Nursing | | #7 = 25 |
| Packed Red | PRBC | | Cardiology, | Yes = 15 | #2 = 10 |
| Blood | | | Resident, | | #4 = 15 |
| Cells | | | Nursing | | #6 = 20 |
| Platelets | PLTS | | Cardiology, | Yes = 15 | Yes = 20 |
| | | | Resident, | | |
| | | | Nursing | | |
| Fresh | FFP | | Cardiology, | Yes = 15 | Yes = 20 |
| Frozen | | | Resident, | | |
| Plasma | | | Nursing | | |
| Aprotinin | APR | | Cardiology, | Yes = 15 | Yes = 15 |
| | | | Resident, | | |
| | | | Nursing | | |

TABLE D

| | | | | Severity | Severity | |
|---|---|---|---|---|---|---|
| | | Pre-bypass | Post-bypass | Scoring | Scoring | |
| Bolus Drugs | ID | Accessibility | Accessibility | Pre-Bypass | Post-Bypass | Note |
| Epinephrine | EPI | Cardiology, | Cardiology, | Yes = 15 | Yes = 25 | |
| | | Resident | Resident, | | | |
| | | | Nursing | | | |
| Calcium | CA | Cardiology | Cardiology, | Yes = 10 | #600 = 10 | Display |
| | | | Resident, | | #800 OR ABOVE = 20 | only if |
| | | | Nursing | | | abnormal |
| | | | | | | parameters |
| | | | | | | are met |
| Bicarb | HCO3 | Cardiology, | Cardiology, | Yes = 20 | Yes = 10 | Display |
| | | Resident, | Resident, | | | only if |
| | | Nursing | Nursing | | | abnormal |
| Ephedrine | EPH | | Resident | | #15 = 5 | Display |
| | | | | | #25 = 10 | only if |
| | | | | | #30 and ABOVE = 20 | abnormal |
| Phenylephrine | NEO | Cardiology | Cardiology, | Yes = 20 | #200 = 5 | Display |
| | | | Resident, | | #300 = 10 | only if |
| | | | Nursing | | #400 and ABOVE = 20 | abnormal |
| Metanephrine | META | | Cardiology, | Yes = 20 | #5 = 5 | Display |
| | | | Resident | | #10 = 10 | only if |
| | | | | | | abnormal |
| Atropine | ATP | Cardiology, | Cardiology, | Yes = 20 | #5 = 10 | Display |
| | | Resident, | Resident, | | #9 and ABOVE = 20 | only if |
| | | Nursing | Nursing | | | abnormal |

TABLE E

| | | Pre-bypass | Post-bypass | Severity Scoring | Severity Scoring |
|---|---|---|---|---|---|
| Drips | ID | Accessibility | Accessibility | Pre-Bypass | Post-Bypass |
| Dopamine | DA | Cardiology | Cardiology, | #3.1 mcq/kg/min | #3.1 = 5 |
| | | | Resident, | and above = 20 | #5 = 15 |
| | | | Nursing | | #7 = 20 |
| | | | | | #9 and ABOVE = 25 |
| Dobutamine | DBT | Cardiology | Cardiology, | | |
| | | | Resident, | | |
| | | | Nursing | | |
| Epinephrine | EPI | Cardiology, | Cardiology, | Yes = 20 | Yes = 25 |
| | | Resident, | Resident, | | |
| | | Nursing | Nursing | | |
| Metanephrine | META | | Cardiology, | Yes = 20 | Yes = 25 |
| | | | Resident, | | |
| | | | Nursing | | |
| Levophed | NOREPI | Cardiology, | Cardiology, | #10 DROPS = 15 | #10 DROPS = 15 |
| | | Resident | Resident, | #20 = 20 | #20 = 20 |
| | | | Nursing | #25 and ABOVE = 25 | #25 and ABOVE = 25 |
| Inocor | INOCOR | Cardiology | Cardiology, | #3.1 = 15 | #3.1 = 15 |
| | | | Resident, | #5 = 20 | #5 = 20 |
| | | | Nursing | #7 = 25 | #7 = 25 |
| | | | | #9 and ABOVE = 30 | #9 and ABOVE = 30 |

TABLE F

| | | Pre-bypass | Post-bypass | Severity Scoring | Severity Scoring |
|---|---|---|---|---|---|
| Drips | ID | Accessibility | Accessibility | Pre-Bypass | Post-Bypass |
| Sodium | SNP | | Cardiology, | #10 DROPS = 15 | #10 DROPS = 15 |
| Nitropursside | | | Resident, | #20 = 20 | #20 = 20 |
| | | | Nursing | #25 and ABOVE = 25 | #25 and ABOVE = 25 |
| Nitroglycerine | NTG | Cardiology, | Cardiology, | #10 DROPS = 15 | #10 DROPS = 15 |
| | | Resident | Resident, | #20 = 20 | #20 = 20 |
| | | | Nursing | #25 and ABOVE = 25 | #25 and ABOVE = 25 |
| Esmolol | ESMO | | Cardiology, | #10 DROPS = 15 | #10 DROPS = 15 |
| | | | Resident, | #20 = 20 | #20 = 20 |
| | | | Nursing | #25 and ABOVE = 25 | #25 and ABOVE = 25 |

TABLE G

| | | | | Severity | Severity |
|---|---|---|---|---|---|
| | | Pre-bypass | Post-bypass | Scoring | Scoring |
| | ID | Accessibility | Accessibility | Pre-Bypass | Post-Bypass |
| Bolus | NEO | | | #200 = 5 | |
| Phenylphrine | | | | #300 = 10 | |
| | | | | #400 and | |
| | | | | ABOVE = 20 | |
| Levophed | LEVO | | | Yes = 20 | |
| Nitro | NTG | | | Yes = 20 | |
| BP-Systolic | BP_S | | | #90 = 5 | |
| | | | | #80 = 10 | |
| | | | | #70 = 20 | |

TABLE H

| | | Pre-bypass | Post-bypass | Severity Scoring | Severity Scoring |
|---|---|---|---|---|---|
| Cardiac | ID | Accessibility | Accessibility | Pre-Bypass | Post-Bypass |
| BP-Systolic | BP-S | Cardiology | Cardiology, | #90 = 10 | #90 = 10 |
| | | | Resident, | #80 = 15 | #80 = 15 |
| BP-Diastolic | BP-D | Cardiology | Cardiology | | |
| BP-mean | BP_M | Cardiology | Cardiology | #65 = 10 | #65 = 10 |
| | | | | #50 = 15 | #50 = 15 |
| PAS | PA_S | Cardiology | Cardiology | #40 = 3 | #40 = 3 |
| | | | | #50 = 10 | #50 = 10 |
| | | | | #60 and ABOVE = 20 | #60 and ABOVE = 20 |
| PAD | PA_D | Cardiology | Cardiology, | #15 = 3 | #15 = 3 |
| | | | Resident, | #20 = 10 | #20 = 10 |
| | | | Nursing | #23 = 15 | #23 = 15 |
| | | | | #25 and ABOVE = 20 | #25 and ABOVE = 20 |
| CVP | CVP | Cardiology | Cardiology, | #15 = 3 | #15 = 3 |
| | | | Resident, | #20 = 10 | #20 = 10 |
| | | | Nursing | #23 = 15 | #23 = 15 |
| | | | | #25 and ABOVE = 20 | #25 and ABOVE = 20 |
| Heart Rate | HR | Cardiology | Cardiology, | #100 = 3 | #100 = 3 |
| | | | Resident, | #120 = 10 | #120 = 10 |
| | | | Nursing | #130 and ABOVE = 20 | #130 and ABOVE = 20 |
| | | | | #60 and BELOW = 10 | #80 = 3 |
| | | | | | #70 = 10 |
| | | | | | #60 and BELOW = 20 |
| Pacemaker | PM | Cardiology, | Cardiology, | Yes = 10 | Yes = 20 |
| | | Resident, | Resident, | | |
| | | Nursing | Nursing | | |
| Intra Aortic | IABP | Cardiology, | Cardiology, | Yes = 20 | Yes = 30 |
| Balloon | | Resident, Nursing | Resident, | | |
| | | | Nursing | | |
| Left | LVAD | Cardiology, | Cardiology, | Yes = 50 | Yes = 50 |
| Ventricular | | Resident, | Resident, | | |
| Assist Device | | Nursing | Nursing | | |

TABLE I

| | | Pre-bypass | Post-bypass | Severity | Severity | |
|---|---|---|---|---|---|---|
| Labs | ID | Accessibility | Accessibility | Scoring Pre-Bypass | Scoring Post-Bypass | Notes |
| PAO2 | PO2 | Resident | Resident, | #250 = 10 | #250 = 10 | All notified |
| | | | Nursing | #150 and BELOW = 20 | #150 and BELOW = 20 | if abnormal |
| PACO2 | PCO2 | Resident | Resident, | #45 and ABOVE = 10 | #45 and ABOVE = 10 | All notified |
| | | | Nursing | | | if abnormal |
| pH | PH | Resident | Resident, | #7.3 and BELOW = 10 | #7.3 and BELOW = 10 | All notified |
| | | | Nursing | | | if abnormal |
| Sodium | NA | Resident | Resident, | | | |
| | | | Nursing | | | |
| Potassium | K | Cardiology | Cardiology, | #5.0 and ABOVE = 15 | #5.0 and ABOVE = 15 | All notified |
| | | | Resident, | | | if abnormal |
| | | | Nursing | | | |
| HCT | HCT | Cardiology, | Cardiology, | #32 and BELOW = 15 | #32 and BELOW = 15 | |
| | | Resident, | Resident, | | | |
| | | Nursing | Nursing | | | |
| Calcium | CA | Resident | Resident | | #1.0 = 5 | All notified |
| | | | | | #0.8 = 10 | if abnormal |

TABLE J

| | | Pre-bypass | Post-bypass | Severity Scoring | Severity Scoring |
|---|---|---|---|---|---|
| Respiratory | ID | Accessibility | Accessibility | Pre-Bypass | Post-Bypass |
| PIP | PIP | | | #35 = 3 | #35 = 3 |
| | | | | #40 = 5 | #40 = 5 |
| | | | | #45 = 10 | #45 = 10 |
| | | | | #50 and ABOVE = 15 | #50 and ABOVE = 15 |
| SAT | SAT | | Cardiology, | #95 = 10 | #95 = 10 |
| | | | Resident | #92 = 15 | #92 = 15 |
| | | | | #90 and BELOW = 15 | #90 and BELOW = 15 |
| Resp. Rate | RR | | Resident, | #14 = 3 | #14 = 3 |
| | | | Nursing | #16 = 6 | #16 = 6 |
| | | | | #18 and ABOVE = 8 | #18 and ABOVE = 8 |

1. A method for inferring a patient's clinical status in the course of a treatment, comprising:
accessing a patient's data; and
identifying at least one abnormal event from the patient's data by applying a scoring system for inferring a patient's clinical status, wherein the scoring system comprises a first scoring scheme for identifying from patient data at least one abnormal event occurring prior to at least one milestone event, and a second scoring scheme for identifying from patient data at least one abnormal event occurring after the at least one milestone event.

2. The method of claim 1, wherein the patient's data comprise pre-operative data, and wherein identification of the at least one abnormal event is based, at least in part, on the pre-operative data.

3. The method of claim 2, wherein the pre-operative data comprise at least one of demographic data and clinical data.

4. The method of claim 1, wherein the patient's data comprise operative data, wherein the operative data comprise data concerning at least one of:
a patient's vital signs,
anesthetics delivered,
ventilation parameters,
drugs delivered,
laboratory results,
intravenous lines attached to the patient,
devices used, and
beginning and start times of events,
and wherein identification of the at least one abnormal event is based, at least in part, on the operative data.

5. The method of claim 1, wherein the at least one abnormal event is identified from a group of abnormal events consisting of abnormal events related to hemodynamics, abnormal events related to laboratory results, and abnormal events indicative of the severity of the patient's condition.

6. The method of claim 1, further comprising producing a briefing comprising at least one abnormal event identified in connection with the patient's treatment.

7. The method of claim 6, wherein the briefing is a multimedia briefing, and wherein the multimedia briefing is made available on a patient post-operative briefing interface comprising controls for a user to control presentation of the multimedia briefing.

8. The method of claim 7, wherein the patient post-operative briefing interface comprises a graphical presentation, in a time-dependent format, of at least one of an abnormal event identified in connection with the patient's treatment and a milestone event.

9. The method of claim 6, wherein the briefing is produced for a target audience.

10. The method of claim 1, wherein at least one scoring scheme assigns a score to abnormal patient data that is based on the severity of the patient's condition, as reflected in the patient's data.

11. The method of claim 1, wherein the patient's data is collected in connection with cardiac bypass surgery, wherein the at least one milestone event is the cardiac bypass, and wherein the first scoring scheme is applied to data occurring prior to the cardiac bypass, and the second scoring scheme is applied to data occurring after the cardiac bypass.

12. The method of claim 1, wherein identification of the at least one abnormal event is based, at least in part, on at least one of:
a duration of the treatment or a portion thereof,
whether blood products were administered,
quantities of blood products administered,
whether drugs were administered, and
quantities of drugs administered.

13. The method of claim 1, further comprising identifying at least one abnormal hemodynamic event by applying temporal abstraction to the patient's operative data that have been scored in accordance with the scoring system for inferring a patient's clinical status.

14. The method of claim 13, wherein temporal abstraction comprises applying a sliding scale average over a window of a predefined number of consecutively-occurring operative data that have been scored in accordance with the scoring system for inferring a patient's clinical status.

15. The method of claim 1, further comprising identifying drugs and quantities thereof administered in connection with an abnormal event, and noting, in a briefing, the drugs and quantities thereof administered.

16. The method of claim 1, further comprising linking a milestone event with one or more abnormal events occurring within a predefined period of time of the milestone event, and noting, in a briefing, whether the one or more abnormal events occurred within a predefined period of time of the milestone event.

17. The method of claim 1, further comprising monitoring a patient's treatment, and displaying an operation status monitor interface comprising a patient summary for at least one patient undergoing a treatment.

18. The method of claim 17, wherein the operation status monitor interface comprises a graphical presentation of the patient's treatment status in a time-dependent format.

19. The method of claim 17, further comprising:
producing a briefing comprising at least one abnormal event identified in connection with the patient's treatment; and
transferring an incidence of the patient from the operation status monitor interface to a patient post-operative briefing interface which indicates that a briefing for the patient is available for review.

20. A method for inferring a patient's clinical status in the course of a treatment, comprising:
accessing a patient's pre-operative and operative data;
identifying from the patient's pre-operative and operative data at least one abnormal event indicative of the severity of a patient's condition, by applying a scoring system for inferring a patient's clinical status, wherein the scoring system comprises a first scoring scheme for identifying from patient data at least one abnormal event occurring prior to at least one milestone event, and a second scoring scheme for identifying from patient data at least one abnormal event occurring after the at least one milestone event; and
producing a briefing comprising at least one abnormal event identified in connection with the patient's treatment.

21. A method for inferring a patient's clinical status in the course of a treatment, comprising:
monitoring a patient's treatment;
displaying an operation status monitor interface comprising a patient summary for at least one patient undergoing a treatment;
accessing the patient's pre-operative and operative data;
identifying abnormal events from the patient's pre-operative and operative data by applying a scoring system for inferring a patient's clinical status;
producing automatically, at the end of the patient's treatment, a briefing comprising at least one abnormal event identified in connection with the patient's treatment; and
transferring an incidence of the patient from the operation status monitor interface to a patient post-operative briefing interface which indicates that a briefing for the patient is available for review.

22. The method of claim 21, wherein the briefing is a multimedia briefing, and wherein the multimedia briefing is made available on a patient post-operative briefing interface comprising controls for a user to control presentation of the multimedia briefing.
